(19) United States
(12) Patent Application Publication
(10) Pub. No.: US 2022/0354341 A1
(43) Pub. Date: Nov. 10, 2022

(54) AUXILIARY OPERATION STRUCTURE OF ENDOSCOPE

(71) Applicant: Ming-Yao CHEN, Taipei, Taiwan 103 (CN)

(72) Inventor: Ming-Yao CHEN, Taipei City (TW)

(21) Appl. No.: 17/762,998

(22) PCT Filed: Sep. 27, 2019
(86) PCT No.: PCT/CN2019/000191

§ 371 Date: Mar. 23, 2022

(51) Int. Cl. A61B 1/00; A61B 1/05; A61B 1/06

(52) U.S. Cl. CPC A61B 1/00042; A61B 1/05; A61B 1/0016; A61B 1/06; A61B 1/00045; A61B 1/00016; A61B 1/00018; A61B 1/00124; A61B 1/0014

(57) ABSTRACT

An auxiliary operation structure (1) for an endoscope, comprising: a control module (10), a human-machine interface module (30) and a clamping module (12). A plurality of pressing units (P1, P2, P3, P4, P5, P6), a plurality of dial driving units (D1, D2) and a plurality of knob driving units (S1, S2) of the control module (10) are connected to a plurality of control units on an operation module (21) of an endoscope (2) of the control module (10). With the auxiliary operation structure (1) for the endoscope (2), a physician can control a joystick (15) via the hands to perform manipulation, such that the operation module (21) of the endoscope (2) can be controlled and operated easily to perform an invasive examination or a minimally invasive operation by means of the endoscope (2). In an invasive examination or a minimally invasive operation, a physician is not required to hold the operation module (21) of the endoscope (2) with the hands, avoiding excessive load on the wrists or arms of the physician and occupational injury thereto.

### FIELD OF THE INVENTION

The present invention is related to a technical field of surgical medical apparatuses and instruments, especially to an auxiliary operation structure for an endoscope.

### BACKGROUND OF THE INVENTION

With the advancement of medical instruments and technologies, an endoscope, such as a gastroscopy and a colonoscopy, not only can be used as an invasive examination instrument, but it also can be used for a minimally invasive surgery. For example, if the tumor invasion depth does not exceed a submucosa and there is no lymphatic metastasis, the endoscope can be used for local excision, and thus avoid the pain resulted from traditional surgical treatment.

FIG. 1A shows a perspective view of a conventional endoscope, and FIG. 1B shows another perspective view of the conventional endoscope. In more detail, the endoscope 1′ shown in FIGS. 1A and 1B is a gastroscopy, and its structure includes an operation module 11′, a cable set 12′, and a pipe 13′. The cable set 12′ is connected between the operation module 11′ and a host (not shown). In addition, the pipe 13′ has one end connected with the operation module 11′, and has another end provided with a lighting unit and a camera unit. According to the conventional common design, the operation module 11′ of the endoscope 1′ is provided with a plurality of control units, including an image fixing button F1′, a first function button F2′, a second function button F3′, a third function button F4′, an attraction-activating button F5′, a fluid delivery button F6′, an instrument insertion channel F7′, a first fixing knob F8′, a first angle adjustment dial F9′, a second fixing knob F10′, and a second angle adjustment dial F11′.

When using the endoscope 1′, a physician will hold the operating module 11′ with one hand (for example, his right hand), and use the other hand (for example, his left hand) as a transmission mechanism for the pipe 13′. Briefly, the physician uses his left hand to insert the pipe 13′ into a patient's body, and then uses the lighting unit and the camera unit at the front end of the pipe 13′ to capture an image of a site to be examined or operated on in the patient's body. Specifically, when the pipe 13′ is placed in the patient's body, the physician must operate the plurality of control units on the operating module 11′. For example, when the physician turns the first angle adjustment dial F9′ to move the front end of the pipe 13′ leftward/rightward. On the other hand, when the physician turns the second angle adjustment dial F11′ to move the front end of the pipe 13′ upward/downward. Further, after the front end of the pipe 13′ is gradually fed and moved to the site to undergo a surgical operation, the physician can then insert a surgical instrument into the site undergoing a surgical operation through the instrument insertion channel F7′, and then use the surgical instrument to perform a minimally invasive surgical treatment on the site undergoing a surgical operation.

It can be understood from above description that when the physician operates the endoscope 1′ shown in FIG. 1A and FIG. 1B, his right hand must continuously hold and control the operation module 11′ of the endoscope 1 ‘ until the front end of the pipe 13’ is gradually fed to a target site in the patient's body (for example, the site undergoing a surgical operation on or the site to be examined). In the case that when the operation module 11′ is held for a long period of time, the physician will inevitably has a sore wrist and/or arm, and in severe cases, the physician's wrist and/or arm would be injured.

As can be understood from above description, there is a need to design a set of auxiliary mechanism to be combined with the endoscope 1′ as shown in FIG. 1, and the physician can use the endoscope 1′ to complete an invasive examination or a minimally invasive surgery by operating the set of auxiliary mechanism instead of using hands to hold the operating module 11′ and the pipe 13′. In view of this, the inventor of the present invention has made great efforts to research, and finally has developed and completed an auxiliary operation structure for an endoscope of the present invention.

### SUMMARY OF THE INVENTION

The main object of the present invention is to provide an auxiliary operation structure for an endoscope. When using the auxiliary operation structure of the endoscope of the present invention, a physician can easily control the control module of the endoscope by controlling the operation module with his two hands, so as to operate the endoscope to complete an invasive examination or a minimally invasive surgery. In the process of invasive examination or a minimally invasive surgery, the physician do not need to hold the control module of the endoscope by his hands, and thus it will not cause any burden or damage to the physician's wrist or arm.

On the other hand, for the physician who operates the endoscope, in the case of using the auxiliary operation structure of the endoscope of the present invention, the physician can intuitively operate the endoscope through the controller, thereby completing an invasive examination or a minimally invasive surgery smoothly.

In order to fulfill above objects, the present invention provides an auxiliary operation structure for an endoscope, comprising a control module, a human-machine interface module and a clamping module;

the control module includes an endoscope, a plurality of pressing units, a plurality of dial driving units and a plurality of knob driving units; wherein the endoscope includes an operation module, a cable set, and a pipe, and the pipe has one end connected with the operation module, and the pipe has another end provided with a lighting unit and a camera unit, and the operation module is provided with a plurality of buttons, a plurality of dials, and a plurality of knobs; wherein the plurality of pressing units is connected with the plurality of buttons of the operation module; the plurality of dial driving units is connected with the plurality of dials of the operation module; the plurality of knob driving units is connected with the plurality of knobs of the operation module;

the man-machine interface module includes a host, a display screen and a controller, and the host is signally connected with the display screen and the controller, and the host is wirely or wirelessly connected with the plurality of pressing units, the plurality of dial driving units and the plurality of knob driving units, and the cable set of the endoscope is connected between the operation module and the host;

the clamping module is wirely or wirelessly connected with the host to have the pipe to be passed through the clamping module and to be stabilized, so as to control the pipe to be moved forward and backward under the control of the controller, so that the pipe has a front end facing and movable forward and backward in relation to a part of a body of a patient to undergoing a medical examination or a surgical operation;

wherein, under the control of the controller, each pressing unit is driven to correspondingly press one button, and each dial driving unit is driven to correspondingly rotate one dial, and the knob driving unit is driven to correspondingly rotate one knob.

In an embodiment, the controller is selected from joystick, mouse, keyboard, touch screen, handlebar, steering wheel or a combination thereof.

In an embodiment, the auxiliary operation structure for an endoscope of the present invention further comprises a box for accommodating the endoscope, the plurality of pressing units, the plurality of dial driving units and the plurality of knob driving units therein, and the front end of the pipe of the endoscope and a main electrical connection end of the cable set are exposed outside the box.

In an embodiment, a motor is provided inside the clamping module, and the motor has a rotating shaft extending out to form an extension shaft, and a transmission wheel is provided on the extension shaft; the pipe enters into the clamping module through an inlet of the clamping module, and the pipe is loaded on the transmission wheel in an inner part of the clamping module, and the pipe contacts a roller fixed inside the clamping module above the inner part of the clamping module, and the pipe leaves from the clamping module through an outlet of the clamping module, such that the pipe is movably clamped by the transmission wheel and the roller, and the transmission wheel is rotated in different directions to drive the pipe to stably move forward and backward.

In the embodiment of the auxiliary operation structure for an endoscope of the present invention, the plurality of buttons includes an image fixing button, a first function button, a second function button, a third function button, an attraction-activating button, and a fluid delivery button.

In the embodiment of the auxiliary operation structure for an endoscope of the present invention, the plurality of pressing units include:

a first pressing unit, connected with the image fixing button, for pressing the image fixing button based on the control of the controller;
a second pressing unit, connected with the first function button, for pressing the first function button based on the control of the controller;
a third pressing unit, connected with the second function button, for pressing the second function button based on the control of the controller;
a fourth pressing unit, connected with the third function button, for pressing the third function button based on the control of the controller;
a fifth pressing unit, connected with the attraction-activating button, for pressing the attraction-activating button based on the control of the controller; and a sixth pressing unit, connected with the fluid delivery button, for pressing the fluid delivery button based on the control of the controller.

In the embodiment of the auxiliary operation structure for an endoscope of the present invention, the first pressing unit, the second pressing unit, the third pressing unit, the fourth pressing unit, the fifth pressing unit, and the sixth pressing unit are respectively any one of the following: an electric-driven key striker or a cylinder type key striker.

In the embodiment of the auxiliary operation structure for an endoscope of the present invention, the plurality of dials includes a first angle adjustment dial and a second angle adjustment dial.

In the embodiment of the auxiliary operation structure for an endoscope of the present invention, the plurality of dial driving units include:

a first dial driving unit, connected with the first angle adjustment dial, for driving the first angle adjustment dial to rotate through an angle based on the control of the controller; and
a second dial driving unit, connected with the second angle adjustment dial, for driving the second angle adjustment dial to rotate through an angle based on the control of the controller.

In the embodiment of the auxiliary operation structure for an endoscope of the present invention, the first dial driving unit and the second dial driving unit are respectively a rotatable dial assembly, and are driven by a motor to be rotated.

In the embodiment of the auxiliary operation structure for an endoscope of the present invention, the plurality of knobs includes a first fixing knob and a second fixing knob.

In the embodiment of the auxiliary operation structure for an endoscope of the present invention, the plurality of knob driving units includes:

a first knob driving unit, connected with the first fixing knob for driving the first fixing knob to rotate through an angle based on the control of the controller; and
a second knob driving unit, connected with the second fixing knob, for driving the second fixing knob to rotate through an angle based on the control of the controller.

In the embodiment of the auxiliary operation structure for an endoscope of the present invention, the first knob driving unit and the second knob driving unit are respectively a rotatable dial assembly or a conveyor belt, and are rotated by the driving of a motor.

The main advantage of the present invention is that in the case of applying the auxiliary operation structure of the endoscope of the present invention, the physician only needs to control the controller with his two hands, and then he can easily control the control module of the endoscope to operate the control module of the endoscope to performs an invasive examination or a minimally invasive surgery. In the process of invasive examination or minimally invasive surgery, the physician do not need to hold the control module of the endoscope by his hands, and thus it will not cause any burden or damage to the physician's wrist or arm.

On the other hand, for the physician who operates the endoscope, in the case of using the auxiliary operation structure of the endoscope of the present invention, he can intuitively operate the endoscope through the controller, thereby smoothly completing an invasive examination or a minimally invasive surgery.

### BRIEF DESCRIPTION OF DRAWINGS

FIG. 1A shows a perspective view of a conventional endoscope;

FIG. 1B shows another perspective view of the conventional endoscope;

FIG. 2 shows a schematic diagram of an auxiliary operation structure for an endoscope of the present invention in application;

FIG. 3A shows a schematic diagram of a clamping module of the auxiliary operation structure for the endoscope of the present invention;

FIG. 3B shows another schematic diagram of the clamping module of the auxiliary operation structure for the endoscope of the present invention;

FIG. 4 shows a perspective view of an endoscope combined with the auxiliary operation structure for the endoscope of the present invention;

FIG. 5 shows another perspective view of the endoscope combined with the auxiliary operation structure for the endoscope of the present invention;

FIG. 6 shows a perspective view of the auxiliary operation structure of the endoscope for the present invention and the endoscope;

FIG. 7 shows a perspective view of the auxiliary operation structure for the endoscope of the present invention and the endoscope.

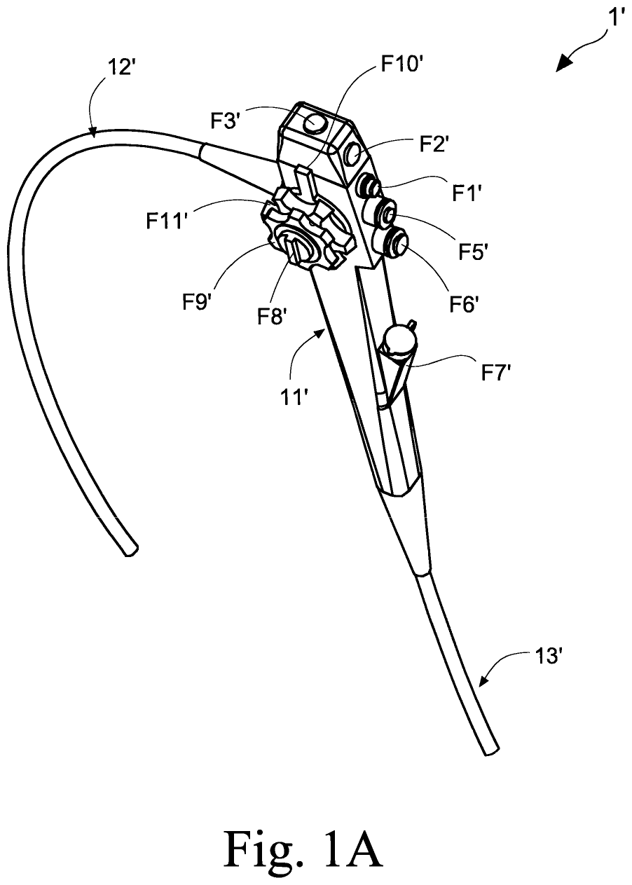

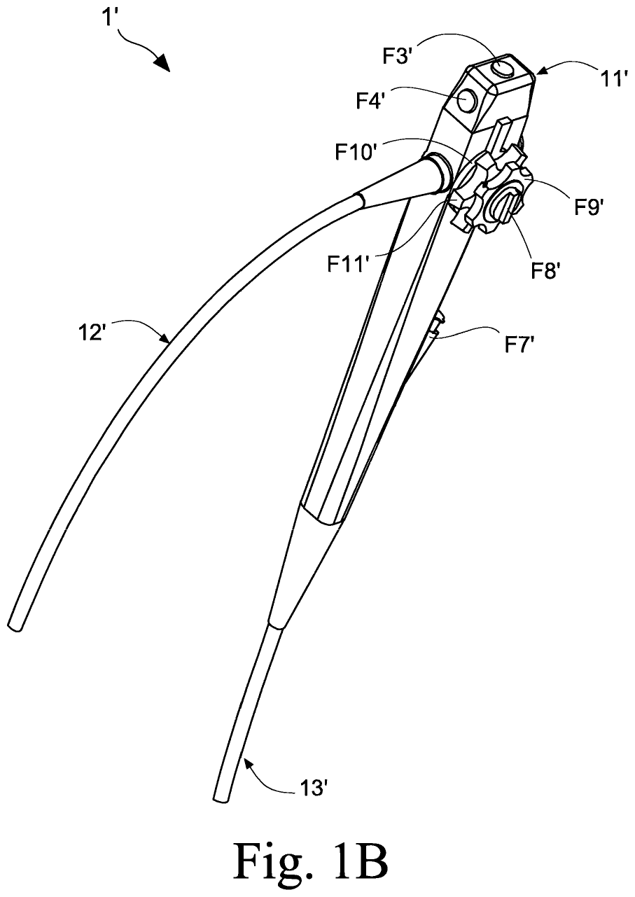

Description of the elements of drawings are as follows: <the present invention> auxiliary operation structure of endoscope: 1; control module: 10; box: 11; clamping module: 12; controller: 15; endoscope: 2; operation module: 21; cable set: 22; pipe: 23; man-machine interface module: 30; display screen: 31; host: 32; transmission wheel: 42; transmission wheel: 44; extension shaft: 45; motor: 46; inlet: 47; outlet: 48; image fixing button: F1; first function button: F2; second function button: F3; third function button: F4; attraction-activating button: F5; fluid delivery button: F6; instrument insertion channel: F7; first fixing knob: F8; first angle adjustment dial: F9; second fixing knob: F10; second angle adjustment dial: F11; first pressing unit: P1; second pressing unit: P2; third pressing unit: P3; fourth pressing unit: P4; fifth pressing unit: P5; sixth pressing unit: P6; first dial driving unit: D1; first motor: D11; second dial driving unit: D2; second motor: D21; first knob driving unit: S1; third motor: S11; second knob drive unit: S2; fourth motor: S21; <prior art> endoscope: 1′; operation module: 11′; cable set: 12′; pipe: 13′; image fixing button: F1; first function button: F2′; second function button: F3′; third function button: F4′; attraction-activating button: F5′; fluid delivery button: F6′; instrument insertion channel: F7; first fixing knob: F8; first angle adjustment dial: F9′; second fixing knob: F10′; second angle adjustment dial: F11′.

### DETAILED DESCRIPTIONS OF PREFERRED EMBODIMENTS

In order to describe the auxiliary operation structure for an endoscope proposed according to the present invention more clearly, the preferred embodiments of the present invention will be described in detail below with reference to the drawings.

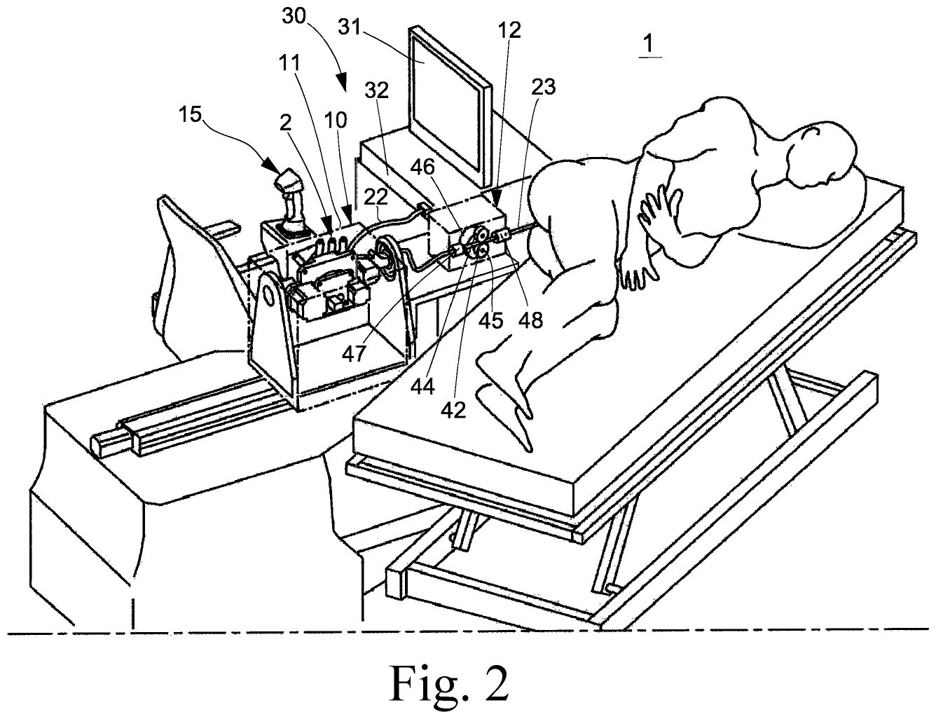

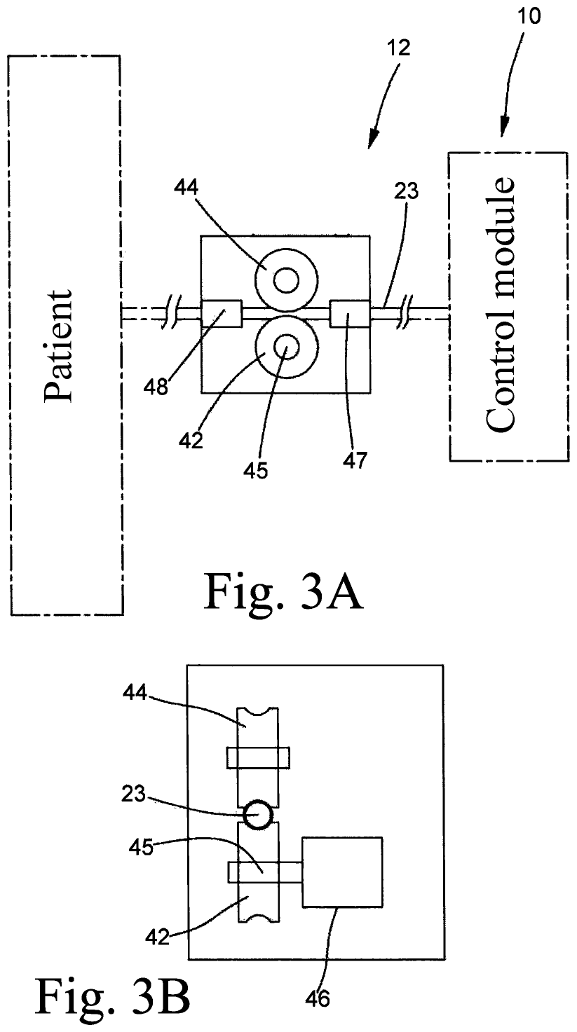

FIG. 2 shows a schematic diagram of an auxiliary operation structure for an endoscope of the present invention in application; FIG. 3A shows a schematic diagram of a clamping module of the auxiliary operation structure for the endoscope of the present invention;

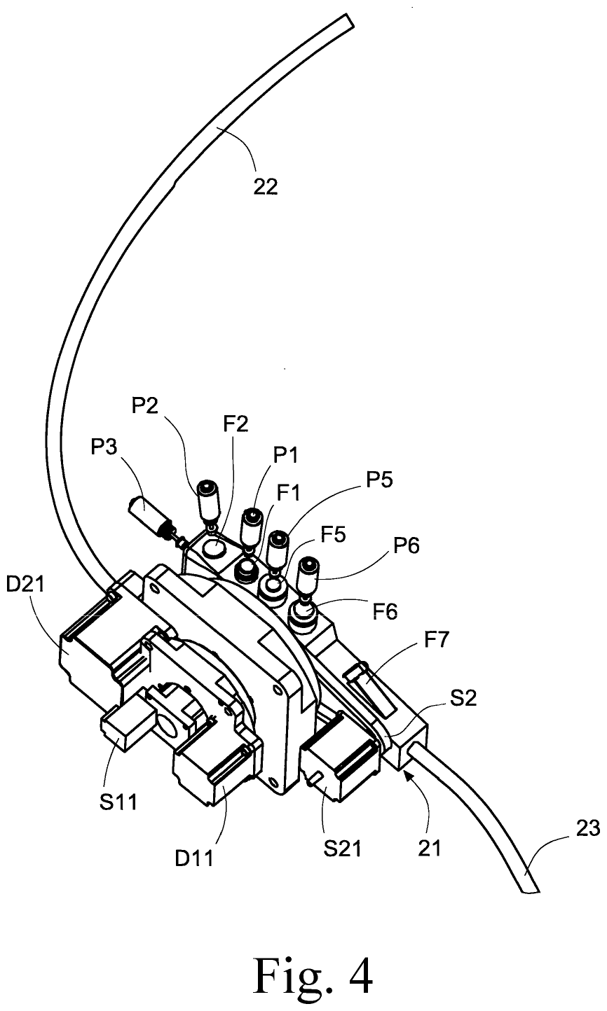

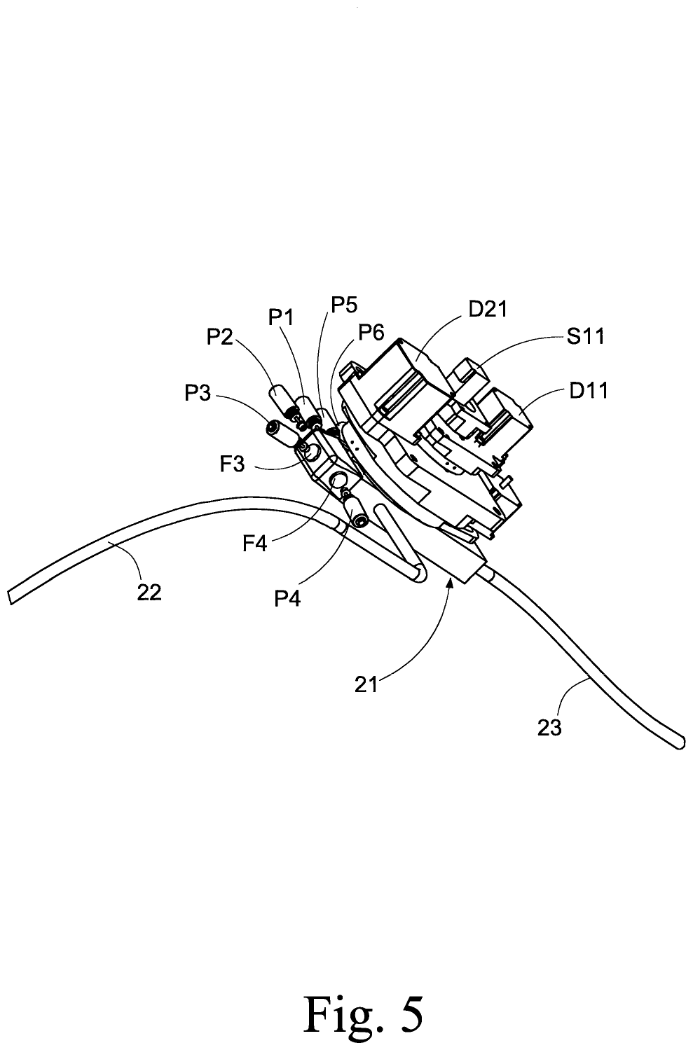

FIG. 3B shows another schematic diagram of the clamping module of the auxiliary operation structure for the endoscope of the present invention; FIG. 4 shows a perspective view of an endoscope combined with the auxiliary operation structure of the endoscope for the present invention; and FIG. 5 shows another perspective view of the endoscope combined with the auxiliary operation structure for the endoscope of the present invention.

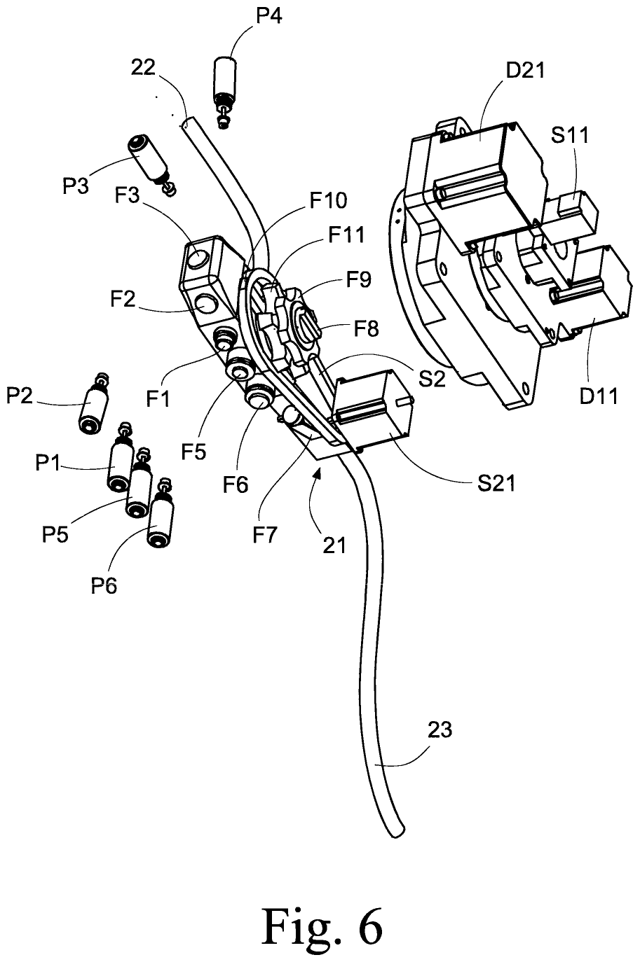

As shown in FIG. 2, FIG. 3A, FIG. 3B, FIG. 4, FIG. 5 and FIG. 6, an auxiliary operation structure 1 for an endoscope of the present invention includes a control module 10, a human-machine interface module 30 and a clamping module 12.

The control module 10 includes an endoscope 2, a plurality of pressing units, a plurality of dial driving units and a plurality of knob driving units; wherein the endoscope 2 includes an operation module 21, a cable set 22, and a pipe 23, and the pipe 23 has one end connected with the operation module 21, and the pipe has another end provided with a lighting unit and a camera unit, and the operation module 21 is provided with a plurality of buttons, a plurality of dials, and a plurality of knobs; wherein the plurality of pressing units is connected with the plurality of buttons of the operation module; the plurality of dial driving units is connected with the plurality of dials of the operation module; the plurality of knob driving units is connected with the plurality of knobs of the operation module.

The man-machine interface module 30 includes a host 32, a display screen 31 and a controller 15, and the host 32 is signally connected with the display screen 31 and the controller 15, and the host 32 is wirely or wirelessly connected with the plurality of pressing units, the plurality of dial driving units and the plurality of knob driving units, and the cable set 22 of the endoscope 2 is connected between the operation module 21 and the host 32.

The clamping module 12 is provided between the control module 10 and a patient, and is signally connected with the control module 10, and is connected with the host 32 via the control module 10 in a wirely or wireless manner, to have the pipe 23 to be passed through the clamping module 12 and to be stabilized, so as to control the pipe 23 to be moved forward and backward under the control of the controller 15, so that the pipe 23 has a front end facing and movable forward and backward in relation to a part of a body of a patient to undergo a medical examination or a surgical operation.

When the controller 15 is operated, the controller 15 transmits a controlling signal to the host 32, and the plurality of pressing units, the plurality of dial driving units and the plurality of knob driving units are wirely or wirelessly connected with controller 15 through the host 32, and thus the host 32 can send out controlling signals to them. Therefore, under the control of the controller 15, each pressing unit is driven to correspondingly press one button, and each dial driving unit is driven to correspondingly rotate one dial, and each knob driving unit is driven to correspondingly rotate one knob.

In an embodiment, FIG. 4, FIG. 5 and FIG. 6 show that the plurality of buttons includes an image fixing button F1, a first function button F2 (FIG. 5), a second function button F3, a third function button F4 (FIG. 5), an attraction-activating button F5, and a fluid delivery button F6. And, the plurality of dials includes a first angle adjustment dial F9 (FIG. 6) and a second angle adjustment dial F11 (FIG. 6), and the plurality of knobs includes a first fixing knob F8 (FIG. 6) and a second fixing knob F10 (FIG. 6).

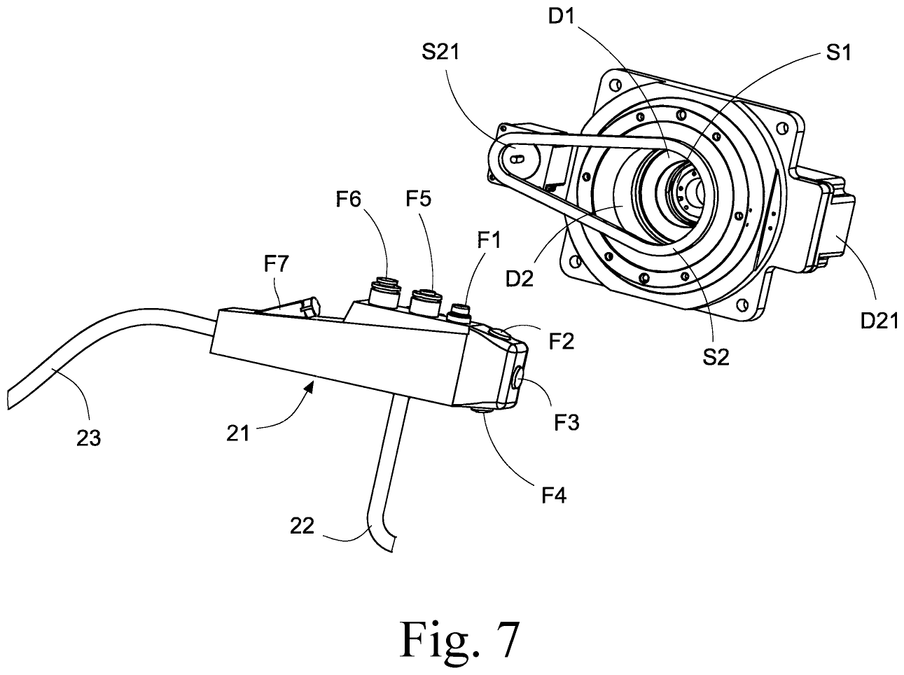

Please refer to FIG. 6 and FIG. 7. FIG. 6 shows a perspective view of the auxiliary operation structure for the endoscope of the present invention and the endoscope; and FIG. 7 shows a perspective view of the auxiliary operation structure for the endoscope of the present invention and the endoscope.

In an embodiment, as shown in FIG. 4, FIG. 5, FIG. 6 and FIG. 7, the plurality of pressing units include a first pressing unit P1, a second pressing unit P2, a third pressing unit P3, a fourth pressing unit P4 (FIG. 6), a fifth pressing unit P5, a sixth pressing unit P6. The first pressing unit P1 is connected with the image fixing button F1 for pressing the image fixing button F1 based on the control of the controller 15. And, the second pressing unit P2 is connected with the first function button F2 for pressing the first function button F2 based on the control of the controller 15. And, a third pressing unit P3 is connected with the second function button F3 for pressing the second function button F3 based on the control of the controller 15.

In accordance with the above description, a fourth pressing unit P4 is connected with the third function button F4 for pressing the third function button F4 based on the control of the controller 15. And, the fifth pressing unit P5 is connected with the attraction-activating button F5 for pressing the attraction-activating button F5 based on the control of the controller 15. Moreover, the sixth pressing unit is connected with the fluid delivery button F6 for pressing the fluid delivery button F6 based on the control of the controller 15. FIGS. 4, 5, 6, and 7 show that the first pressing unit P1, the second pressing unit P2, the third pressing unit P3, the fourth pressing unit P4, the fifth pressing unit P5 and the sixth pressing unit P6 are all cylinder type key strikers. However, in another embodiment, each of the pressing units (P1-P6) also can be an electric-driven key striker.

As for the design of an operation module 21 corresponding to an endoscope 2, in an embodiment, the plurality of dials includes a first angle adjustment dial D1 (FIG. 7) and a second angle adjustment dial D2 (FIG. 7). As shown in FIGS. 4, 5, 6 and 7, the first dial driving unit D1 is connected with the first angle adjustment dial F9 for driving the first angle adjustment dial F9 to rotate through an angle based on the control of the controller 15. And the second dial driving unit D2 is connected with the second angle adjustment dial F11 for driving the second angle adjustment dial F11 to rotate through an angle based on the control of the controller 15. FIGS. 4, 5, 6 and 7 show that the first dial driving unit D1 and the second dial driving unit D2 are respectively a rotatable dial assembly, and are respectively driven by a first motor D11 (FIG. 6) and a second motor D21 (FIG. 7) to be rotated.

In more detail, the plurality of knob driving units includes a first knob driving unit S1 (FIG. 7) and a second driving unit S2 (FIG. 7). As shown in FIGS. 4, 5, 6 and 7, the first knob driving unit S1 is connected with the first fixing knob F8 (FIG. 6) for driving the first fixing knob F8 to rotate through an angle based on the control of the controller 15. And the second knob driving unit S2 is connected with the second fixing knob F10 for driving the second fixing knob F10 to rotate through an angle based on the control of the controller 15. It can be understood from FIG. 4, FIG. 5, FIG. 6, and FIG. 7 that the first knob driving unit S1 is a rotatable dial assembly, and the second knob driving unit S2 is a conveyor belt, and the two are respectively driven by a third motor S11 and a fourth motor S21 to be rotated.

It should be noted that even though FIG. 2 shows that the controller 15 is a joystick device, it should be understood that the implementable mode of the controller 15 should not be limited to a joystick device. For example, the controller 15 may be selected from joystick, mouse, keyboard, touch screen, handlebar, steering wheel or a combination thereof, but not limited thereto.

In practical application of the present invention, the controller 15 is wirely or wirelessly connected with the plurality of pressing units (P1-P6), the plurality of dial driving units (D1-D2) and the plurality of knob driving units (S1-S2) through the host 32. In this way, a user (that is, a physician) controls each of the pressing units (P1-P6) by operating the controller 15 to correspondingly press the buttons (F1-F6) on the operation module 21 of the endoscope 2, so as to drive each dial driving units (D1-D2) to correspondingly rotate one of the dials (F9, F11) on the operation module 21, and/or to drive each of the knob driving units (S1-S2) to correspondingly rotate one of the knobs (F8, F10).

Please continue to refer to FIG. 2. In the case of using the auxiliary operation structure 1 for the endoscope of the present invention, the endoscope 2, the plurality of pressing units, the plurality of dial driving units and the plurality of knob driving units are accommodated in a box 11, and the front end of the pipe 23 of the endoscope 2 and a main electrical connection end of the cable set 22 are exposed outside the box 11. Further, the physician can sit in front of a control table, and a display screen 31 and a host 32 are arranged on the control table. Then, the physician can easily control the endoscope 2 by using his hands to operate the controller 15 placed on the control table without causing any burden or injury to the physician's wrist or arm.

Furthermore, the physician can control the controller 15, so as to have the pipe 23 passing through the clamping module 12 fed by the clamping module 12 to advance toward a target, such as a site in the body of a patient to be examined or a site in the body of the patient undergoing a surgical operation. Then, the physician can use the lighting unit and the camera unit provided at the front end of the pipe 23 to capture an image of the site to be examined or undergoing a surgical operation, and the relevant image will be displayed on the display screen 31 in real time under the control of the host 32. Finally, after the clamping module 12 gradually feeds the front end of the pipe 23 to the site undergoing a surgical operation, the physician can thus move a surgical instrument to the site undergoing a surgical operation through an instrument insertion channel F7, and then use the surgical instrument to perform a minimally invasive surgical treatment on the site undergoing a surgical operation.

In an embodiment, as shown in FIG. 3A and FIG. 3B, a motor 46 is provided inside the clamping module 12, and the motor 46 has a rotating shaft extending out to form an extension shaft 45, and a transmission wheel 42 is provided on the extension shaft 45. The pipe 23 enters into the clamping module 12 through an inlet 47 of the clamping module 12, and the pipe 23 is loaded on the transmission wheel 42 in an inner part of the clamping module 12, and the pipe 23 contacts a roller 44 fixed inside the clamping module 12 above the inner part of the clamping module 12, and the pipe 23 leaves from the clamping module 12 through an outlet 48 of the clamping module 12, such that the pipe 23 is movably clamped by the transmission wheel 42 and the roller 44, and the transmission wheel 42 is rotated in different directions to drive the pipe 23 to stably move forward and backward, such that the pipe 23 is introduced into or withdrawn from the site undergoing a surgical operation.

The above description is only illustrative rather than restrictive for the present invention, and many modifications, variations or equivalents may be made by those skilled in the art without departing from the teachings disclosed hereinabove, but all will be regarded to fall into the scope defined by the appended claims.

What is claimed is:

1. An auxiliary operation structure for an endoscope, comprising a control module, a human-machine interface module and a clamping module;
the control module includes an endoscope, a plurality of pressing units, a plurality of dial driving units and a plurality of knob driving units; wherein the endoscope includes an operation module, a cable set, and a pipe, and the pipe has one end connected with the operation module, and the pipe has another end provided with a lighting unit and a camera unit, and the operation module is provided with a plurality of buttons, a plurality of dials, and a plurality of knobs; wherein the plurality of pressing units is connected with the plurality of buttons of the operation module; the plurality of dial driving units is connected with the plurality of dials of the operation module; the plurality of knob driving units is connected with the plurality of knobs of the operation module;
the man-machine interface module includes a host, a display screen and a controller, and the host is signally connected with the display screen and the controller, and the host is wirely or wirelessly connected with the plurality of pressing units, the plurality of dial driving units and the plurality of knob driving units, and the cable set of the endoscope is connected between the operation module and the host;
the clamping module is wirely or wirelessly connected with the host to have the pipe to be passed through the clamping module and to be stabilized, so as to control the pipe to be moved forward and backward under the control of the controller, so that the pipe has a front end facing and movable forward and backward in relation to a part of a body of a patient to undergo a medical examination or a surgical operation;
wherein, under the control of the controller, each pressing unit is driven to correspondingly press one button, and each dial driving unit is driven to correspondingly rotate one dial, and the knob driving unit is driven to correspondingly rotate one knob.

2. The auxiliary operation structure for an endoscope according to claim 1, wherein the controller is selected from joystick, mouse, keyboard, touch screen, handlebar, steering wheel or a combination thereof.

3. The auxiliary operation structure for an endoscope according to claim 1, further comprising a box for accommodating the endoscope, the plurality of pressing units, the plurality of dial driving units and the plurality of knob driving units therein, and the front end of the pipe of the endoscope and a main electrical connection end of the cable set are exposed outside the box.

4. The auxiliary operation structure for an endoscope according to claim 1, wherein a motor is provided inside the clamping module, and the motor has a rotating shaft extending out to form an extension shaft, and a transmission wheel is provided on the extension shaft; the pipe enters into the clamping module through an inlet of the clamping module, and the pipe is loaded on the transmission wheel in an inner part of the clamping module, and the pipe contacts a roller fixed inside the clamping module above the inner part of the clamping module, and the pipe leaves from the clamping module through an outlet of the clamping module, such that the pipe is movably clamped by the transmission wheel and the roller, and the transmission wheel is rotated in different directions to drive the pipe to stably move forward and backward.

5. The auxiliary operation structure for an endoscope according to claim 1, wherein the plurality of buttons includes an image fixing button, a first function button, a second function button, a third function button, an attraction-activating button, and a fluid delivery button; the plurality of pressing units includes:
a first pressing unit, connected with the image fixing button, for pressing the image fixing button based on the control of the controller;
a second pressing unit, connected with the first function button, for pressing the first function button based on the control of the controller;
a third pressing unit, connected with the second function button, for pressing the second function button based on the control of the controller;
a fourth pressing unit, connected with the third function button, for pressing the third function button based on the control of the controller;
a fifth pressing unit, connected with the attraction-activating button, for pressing the attraction-activating button based on the control of the controller; and
a sixth pressing unit, connected with the fluid delivery button, for pressing the fluid delivery button based on the control of the controller.

6. The auxiliary operation structure for an endoscope according to claim 5, the first pressing unit, the second pressing unit, the third pressing unit, the fourth pressing unit, the fifth pressing unit, and the sixth pressing unit are respectively any one of the following: an electric-driven key striker or a cylinder type key striker.

7. The auxiliary operation structure for an endoscope according to claim 1, wherein the plurality of dials includes a first angle adjustment dial and a second angle adjustment dial;
the plurality of dial driving units includes:
a first dial driving unit, connected with the first angle adjustment dial, for driving the first angle adjustment dial to rotate through an angle based on the control of the controller; and
a second dial driving unit, connected with the second angle adjustment dial, for driving the second angle adjustment dial to rotate through an angle based on the control of the controller;
the first dial driving unit and the second dial driving unit are respectively a rotatable dial assembly, and are driven by a motor to be rotated.

8. The auxiliary operation structure for an endoscope according to claim 1, wherein the plurality of knobs includes a first fixed knob and a second fixed knob;
the plurality of knob driving units includes:
a first knob driving unit, connected with the first fixed knob for driving the first fixed knob to rotate through an angle based on the control of the controller; and
a second knob driving unit, connected with the second fixed knob, for driving the second fixed knob to rotate through an angle based on the control of the controller.

9. The auxiliary operation structure for an endoscope according to claim 1, wherein the first knob driving unit and the second knob driving unit are respectively a rotatable dial assembly or a conveyor belt, and are rotated by the driving of a motor.
